(19) United States
(12) Patent Application Publication
(10) Pub. No.: US 2022/0233495 A1
(43) Pub. Date: Jul. 28, 2022

(54) CANNABINOID FORMULATIONS

(71) Applicant: GW Research Limited, Cambridge (GB)

(72) Inventors: Alan SILCOCK, Cambridge (GB); Jitinder WILKHU, Cambridge (GB)

(21) Appl. No.: 17/615,422

(22) PCT Filed: May 28, 2020
(86) PCT No.: PCT/GB2020/051290

§ 371 Date: Nov. 30, 2021

(30) Foreign Application Priority Data
May 31, 2019 (GB) 1907786.6

(51) Int. Cl. A61K 31/352; A61K 9/16; A61K 31/05

(52) U.S. Cl. CPC A61K 31/352; A61K 9/1635; A61K 9/1652; A61K 9/1694; A61K 31/05

(57) ABSTRACT

The present invention relates to a pharmaceutical formulation containing one or more cannabinoids. Preferably the formulation is a molecular dispersion of one or more cannabinoids in a pH dependent release polymer. Preferably the formulation is able to target delivery of the cannabinoids to specific areas of the digestive system such as the colon or intestines.

### FIELD OF THE INVENTION

The present invention relates to a pharmaceutical formulation containing one or more cannabinoids. Preferably the formulation is a molecular dispersion of one or more cannabinoids in a pH dependant release polymer. Preferably the formulation is able to target delivery of the cannabinoids to specific areas of the digestive system such as the colon or intestines.

### BACKGROUND TO THE INVENTION

Cannabinoids are lipophilic substances that are known to be poorly soluble in water (less than 1 μg/mL). In contrast, and by way of example, cannabidiol (CBD) is soluble in ethanol at 36 mg/mL and the polar solvent dimethyl sulfoxide (DMSO) at 60 mg/mL.

The contemporary use of cannabinoids in medicine has necessitated finding more effective ways of delivering these poorly soluble compounds. In addition to poor aqueous solubility cannabinoids are also known to have limited bioavailability and poor stability in formulations.

If cannabinoids are required to be provided at relatively high doses (in daily amounts of up to 2000 mg) and/or in challenging patient groups, e.g. young children, and/or for particular indications this can create further challenges.

There are currently four commercially available cannabinoid formulations on the market which due to the lack of solubility of cannabinoids utilise alcohol and/or oil based excipients. These are: dronabinol (Marinol®) which is a synthetic tetrahydrocannabinol (THC) which is delivered orally, in sesame oil as capsules; nabilone (Cesamet®) which is a synthetic cannabinoid and an analog of THC and is delivered orally in capsules with povidone and corn starch; nabiximols (Sativex®) a natural extract of cannabinoids, dissolved in ethanol and propylene glycol, containing defined amounts of THC and Cannabidiol (CBD) delivered as a liquid, by way of an oromucosal spray and cannabidiol (Epidiolex®) which is an oral formulation comprising botanically derived purified CBD. The CBD is formulated in sesame oil and further comprises the sweetener sucralose, strawberry flavouring and up to 10% v/v ethanol.

Whilst there is no clear FDA guidance for maximum allowable ethanol concentration in prescription medicines, an article (Ethanol in Liquid Preparations Intended for Children, Paediatrics: Official Journal of The American Academy of Paediatrics, 1984: 73:405), recommends that a Blood Alcohol Concentration (BAC) of 0.25 g/L (250 mg/L) should not be exceeded following a single dose of alcohol containing medications.

Furthermore, the use of oil-based formulations often causes gastrointestinal side effects such as diarrhoea which can be so severe it may cause the patient to discontinue use of the medication.

Alternative approaches to cannabinoid formulations have been suggested.

WO 2015/184127 (Insys) discloses a number of different oral formulations including: an alcohol-free formulation, in which the cannabinoid is formulated in a mix of polyethylene glycol and propylene glycol, optionally with water; a formulation containing alcohol; and a formulation containing lipids. In each of the formulations disclosed, the cannabinoid is a synthetically produced (as opposed to a naturally extracted) cannabidiol. The specification teaches the inclusion of a number of pharmaceutically acceptable excipients such as, antioxidants, sweeteners, enhancers, preservatives, flavouring agents and pH modifiers.

WO 2012/033478 (Murty), discloses Self Emulsifying Drug Delivery Systems (SEDDS) which are said to offer improved administration of cannabinoids. SEDDS generally consist of hard or soft capsules filled with a liquid or a gel that consists of lipophilic active pharmaceutical ingredient (API), oil (to dissolve the API) and a surfactant. Upon contact with gastric fluid, the SEDDS spontaneously emulsify due to the presence of surfactants. Many surfactants, however, are lipid based and interact with lipases in the GIT. This can lead to a reduced capability of the lipid based surfactants to emulsify the API as well as the oil carrier, both reducing bioavailability.

Lipid based formulations are classified according to the Lipid Formulation Classification System (LFCS), Type I formulations are oils which require digestion, Type II formulations are water-insoluble self-emulsifying drug delivery systems (SEDDS), Type III systems are SEDDS or self-micro emulsifying drug delivery systems (SMEDDS) or self-nano emulsifying drug delivery systems (SNEDDS) which contain some water-soluble surfactants and/or co-solvents (Type IIIA) or a greater proportion of water soluble components (Type IIIB). Category Type IV represents a recent trend towards formulations which contain predominantly hydrophilic excipient surfactants and co-solvents.

Table 1, below, is a tabular Lipid Formulation Classification System overview taken from US 2015/111939:

| | Content of formulation (wt.-%) | | | | |
|---|---|---|---|---|---|
| | Type | Type | Type | Type | Type |
| Excipients in formulation | I | II | IIIA | IIIB | IV |
| Oil: triglycerides or mixed mono- | 100 | 40-80 | 40-80 | <20 | — |
| and diglycerides | | | | | |
| Water-insoluble surfactants | — | 20-60 | — | — | 0-20 |
| (HLB <12) | | | | | |
| Water-soluble surfactants | — | — | 20-40 | 20-50 | 30-80 |
| (HLB >12) | | | | | |
| Hydrophilic co-solvent | — | — | 0-40 | 20-50 | 0-50 |

A further description of the Lipid Formulation Classification System can also be found in FABAD J. Pharm. Sci., pages 55-64, 2013.

Drug Development and Industrial Pharmacy (2014), 40, 783-792 discloses the general principals of formulating drugs with poor water solubility. More specifically it discusses the formulation of phenobarbital, a drug with a solubility of 1 mg/ml which is 1000 times more soluble than cannabidiol in water.

It states the presence of co-solvents in the formulations are critical to the stability of the drug, and further states that the biggest limitation of co-solvency is the toxicity of most water miscible co-solvents that have a high potential for increasing drug solubility. It concludes the formulation of this poorly water-soluble drug represents a challenging task for formulation experts.

The microemulsions it teaches are colloidal dispersions, thermodynamically stable systems that are isotropic and have low viscosity. The structure consists in microdomains of lipids or water, stabilised by an interfacial film of surfactant and co-surfactant molecules. They are classified as oil in water or water in oil emulsions and the droplet size is less than 150 nm.

It also discusses the increased interest in S(M)EDDS which are isotropic mixtures of oil, surfactant, co-surfactant and drug. The efficacy of oral formulations of these is stated to depend on many formulation related parameters including: surfactant concentration, oil/surfactant ratio, polarity of the emulsion, droplet size and charge. Additionally, taste is stated to have an important role in compliance.

The formulations developed all comprised surfactant (Cremophor or Labrasol, at 20% w/w), a separate oil phase, (a number of oils were tested which were proprietary forms of: glycerol monocaprylocaprate, caprylic/capric triglyceride, propylene glycol caprylate and propylene glycol dicaprylate/dicaprate were tested, typically at 4% w/w), and a co-surfactant (including Transcutol, PEG 400, glycerol, ethanol and propylene glycol, typically at concentrations between 20 and 35% w/w).

The conclusion was that Phenobarbital could be dissolved easily in a number of microemulsions but the selection of the oil phase was very important.

Additional cannabinoid formulations from the art include:

US2016/0213624, which describes formulations of a hemp oil, and not cannabinoids per se, by emulsification with a surfactant/emulsifier, such as Polysorbate 80. The surfactant/emulsifier is used in an amount of less than 0.02% v/v.

US2016/0184258 which discloses SEDDS formulations, particularly type III formulations which comprise e.g. a Cannabis extract, dissolved in ethanol, an oil base—typically about 35-56%, a surfactant—typically about 28-52%, and a co-solvent—such as ethanol, typically about 7-9%.

International Journal of Pharmaceutics discloses non-ionic microemulsions of THC for parenteral administration using Solutol as a surfactant without the addition of lipids, co-surfactants or other modifiers. The resulting microemulsion contained 0.19% THC and 2.52% (by wt) Solutol.

Pharmacology, Biochemistry and Behaviour 2017, 153, p 69-75 discloses Cremophor/saline (10/90) solutions of THC at concentrations of up to 5 mg/ml THC.

CN103110582 also discloses a cannabinoid containing micro-emulsion containing the following components in percentage by weight: (a) 0.01 wt %-30 wt %/c) cannabinoid; (b) 0.01 wt %-30 wt % of oil phase; (c) 0.01 wt %-60 wt % of surfactant; and; (d) 0.01 wt %-40 wt % of cosurfactant.

“Cannabinoids delivery systems based on supramolecular inclusion complexes and polymeric nanocapsules for treatment of neuropathic pain” (Fanny Astruc-Diaz, Université Claude Bernard) discloses polymeric nanocapsules, in the range of 100 nm, for the delivery of beta-caryophyllene. This document wrongly describes beta-caryophyllene as a cannabinoid, however this compound is a sesquiterpene.

US 2012/231083 (Carley et al) discloses immediate release and delayed release pellets comprising synthetic THC, one such pellet containing: (a) 3.49% w/w Dronabinol; (b) 3.49% w/w Sodium Lauryl Sulfate; (c) 27.91% w/w Neusilin US2; (d) 34.88% w/w Avicel PH101; (e) 5.30% w/w Ethyl cellulose; (f) 1.67% w/w Dibutyl Sebacate.

WO 2008/024490 (Theraquest Biosciences, Inc.) discloses a number of different compositions comprising a cannabinoid agonist and an opioid agonist, including one made up of cannabidiol, naloxone, Eudragit RSPO, Eudragit RLPO and stearyl alcohol.

WO 2019/159174 (Icdpharma Ltd.) discloses a solid solution composition comprising one or more cannabinoids, wherein the solid solution disintegrates or erodes or swells when in contact with body fluids.

WO 2018/035030 (Corr-Jensen Inc.) discloses an extended release fat-soluble active composition which could comprise a range of different actives such as vitamins, carotenoids, polyunsaturated fatty acids and cannabinoids.

Clearly there is a need to have oral formulations (as opposed to injectables which are not designed for, nor indeed suitable for, oral delivery) which are more bioavailable, and which can deliver sufficient amounts of cannabinoids (greater than 0.5%, more preferably still at least 1% by wt) in a patient friendly formulation.

In addition to the problems with the use of ethanol, or an oil-based excipient, in cannabinoid containing oral formulations, the strong bitter taste of cannabinoids provides a further problem which needs to be overcome when producing an oral cannabinoid formulation.

For paediatric products aimed at younger children, it is desirable to have low or no ethanol formulations, preferably dispensed as a syrup, as younger children find it difficult to swallow capsules. They also favour sweet, flavoured products, such as syrups, particularly where the taste of the active agent requires masking.

Cannabinoids are also known to metabolise quickly, particularly when delivered as an oral solution. For example, the cannabinoid cannabidiol (CBD) quickly degrades in the body to 7-hydroxy cannabidiol (7-OH CBD) which then subsequently degrades to 7-carboxy cannabidiol (7-COOH CBD). In the treatment of epilepsy, it is known that the 7-OH metabolite is active but the 7-COOH metabolite (which is the final metabolite) is inactive and as such the rapid degradation from CBD to 7-COOH CBD is unwanted and requires more active to be provided to successfully treat a patient.

Consequently, avoiding or slowing down the metabolism of the cannabinoid would enable a medicament that produces better bioavailability and would allow for lower doses of medicine to be provided.

Specifically delivering drugs to the colon or intestines has been a desirable target for drug delivery systems but thus far have not provided a formulation which comprises the challenging drug substance of cannabinoids.

The approaches for colon specific drug delivery are to utilise excipients that interact with one or more aspects of the gastrointestinal system. In addition, the formulation must be able to resist digestion within the stomach.

An object of the present invention was to develop alternative cannabinoid containing formulations which were gastric resistant and able to deliver cannabinoids to the enteric or colonic areas. Such formulations must provide good bioavailability and stability of the cannabinoid active in order to be viable for drug development.

In one embodiment the invention provides a formulation in the form of a suspension comprising microparticulates which comprise the active agent of a cannabinoid in addition to excipients which enable targeted delivery to the colon or intestines and avoid digestion in the stomach.

In a further embodiment the invention provides a formulation which comprises a granulate. The granulate comprises the cannabinoid microparticulate but may be used to produce alternative dosage forms such as tablets, filled capsules and sprinkles.

### BRIEF SUMMARY OF THE DISCLOSURE

In accordance with a first aspect of the present invention there is provided a microparticulate cannabinoid containing formulation comprising one or more cannabinoids and a pH dependant release polymer.

Preferably the one or more cannabinoids are taken from the group consisting of: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA).

Preferably the one or more cannabinoids are a pure, isolated or synthetic cannabinoid.

Alternatively, the one or more cannabinoids are present as a botanical drug substance.

In a further aspect of the invention the one or more cannabinoids are present as a mixture of a purified, isolated or synthetic cannabinoid and a botanical drug substance.

Preferably the pH dependant release polymer is taken from the group consisting of: a copolymer of methacrylic acid and methacrylate, a copolymer of methacrylic acid and methyl methacrylate (Eudragit), a copolymer of methacrylic acid and ethylacrylate, hydroxypropyl methyl cellulose acetate succinate (HPMCAS), hydroxypropyl methyl cellulose phthalate (HPMCP), polyvinyl acetate phthalate (PVAP), a copolymer of methyl vinyl ether and maleic anhydride, cellulose acetate phthalate (CAP), cellulose acetate butyrate (CAB), cellulose acetate trimellitate (CAT), cellulose acetate succinate (CAS), ethyl cellulose, methyl cellulose, shellac, gellan gum, zein, alginic acid and waxes.

More preferably the pH dependant release polymer is HPMCAS or Eudragit.

More preferably still the pH dependant release polymer is taken from the group consisting of: HPMCAS-L; HPMCAS-M; HPMCAS-H; Eudragit S100; Eudragit L100.

Preferably the microparticulate cannabinoid containing formulation further comprises one or more wetting agents.

More preferably the one or more wetting agents is taken from the group consisting of: poloxamers; poloxamer 188; and sodium carbonate.

In a further embodiment of the invention the formulation further comprises one or more suspending agents.

Preferably the one or more suspending agents are taken from the group consisting of: polysorbate 20; glycerol; and xanthan gum.

In a further embodiment of the invention the formulation further comprises one or more pH buffers.

Preferably the one or more pH buffers are taken from the group consisting of: citric acid; sodium phosphate dibasic; sodium hydroxide; and phosphate buffered saline.

In a further embodiment of the invention the formulation further comprises one or more preservatives.

Preferably the one or more preservatives are taken from the group consisting of: potassium sorbate; and sodium benzoate.

In a further embodiment of the invention the formulation further comprises one or more antioxidants.

Preferably the one or more antioxidants are taken from the group consisting of: butylated hydroxyltoluene; butylated hydroxylanisole; alpha-tocopherol (Vitamin E); ascorbyl palmitate; ascorbic acid; sodium ascorbate; ethylenediamino tetraacetic acid; cysteine hydrochloride; citric acid; sodium citrate; sodium bisulfate; sodium metabisulfite; lecithin; propyl gallate; sodium sulfate; monothioglycerol and mixtures thereof.

In a further embodiment of the invention the formulation further comprises one or more solvents.

Preferably the one or more solvents is taken from the group consisting of: water; ethanol and acetone.

Preferably the one or more cannabinoids are present in an amount of from about 10 to 50 wt %, based on the pharmaceutical formulation, preferably from about 10 to 30 wt %, more preferably from about 20 to 30 wt %.

Preferably the formulation is an oral dosage form selected from the group consisting of a mucoadhesive gel; a tablet; a powder; a liquid gel capsule; a solid capsule; an oral solution; an oral suspension; a granulate; and an extrudate.

In a further aspect of the present invention the microparticulate cannabinoid containing formulation is for use in the treatment of conditions requiring the administration of a neuroprotectant or anti-convulsive medication.

Preferably the formulation is for use in the treatment of seizures.

More preferably the formulation is for use in the treatment of Dravet syndrome, Lennox Gastaut syndrome, myoclonic seizures, juvenile myoclonic epilepsy, refractory epilepsy, schizophrenia, juvenile spasms, West syndrome, infantile spasms, refractory infantile spasms, tuberous sclerosis complex, brain tumours, neuropathic pain, Cannabis use disorder, post-traumatic stress disorder, anxiety, early psychosis, Alzheimer's disease, and autism.

In a second aspect of the present invention there is provided a method of preparing a microparticulate cannabinoid containing formulation according to any of the preceding claims, comprising spray drying the formulation.

In a third aspect of the present invention there is provided a method of preparing a microparticulate cannabinoid containing formulation according to any of the preceding claims, comprising: Preparing a mixture of the cannabinoid and pH dependant release polymer; Producing an intermediate powder blend; Processing the intermediate powder blend through a hot melt extruder; Pelleting the extrudates; and Milling the pellets to 250-500 μm.

Preferably an antioxidant and/or a disintegrant is added after preparing the mixture of the cannabinoid and pH dependant release polymer.

In a fourth aspect of the present invention there is provided method of treating a subject comprising administering a microparticulate cannabinoid containing formulation to the subject.

Preferably the subject is a human.

### BRIEF DESCRIPTION OF THE DRAWINGS

FIG. 1 is a graph depicting the area under the curve (AUC) for the 7-COOH CBD metabolite from the bioavailability study.

### DEFINITIONS

“Cannabinoids” are a group of compounds including the endocannabinoids, the phytocannabinoids and those which are neither endocannabinoids or phytocannabinoids, hereinafter “syntho-cannabinoids”.

“Endocannabinoids” are endogenous cannabinoids, which are high affinity ligands of CB1 and CB2 receptors.

“Phytocannabinoids” are cannabinoids that originate in nature and can be found in the Cannabis plant. The phytocannabinoids can be present in an extract including a botanical drug substance, isolated, or reproduced synthetically.

“Syntho-cannabinoids” are those compounds capable of interacting with the cannabinoid receptors (CB1 and/or CB2) but are not found endogenously or in the Cannabis plant. Examples include WIN 55212 and rimonabant.

An “isolated phytocannabinoid” is one which has been extracted from the Cannabis plant and purified to such an extent that all the additional components such as secondary and minor cannabinoids and the non-cannabinoid fraction have been removed.

A “synthetic cannabinoid” is one which has been produced by chemical synthesis. This term includes modifying an isolated phytocannabinoid, by, for example, forming a pharmaceutically acceptable salt thereof or by the process of producing a pro-drug of a cannabinoid by the addition of one or more groups to the cannabinoid molecule to render the molecule inactive until it is metabolised within the body.

A “substantially pure” cannabinoid is defined as a cannabinoid which is present at greater than 95% (w/w) pure. More preferably greater than 96% (w/w) through 97% (w/w) thorough 98% (w/w) to 99% % (w/w) and greater.

A “highly purified” cannabinoid is defined as a cannabinoid that has been extracted from the Cannabis plant and purified to the extent that other cannabinoids and non-cannabinoid components that are co-extracted with the cannabinoids have been substantially removed, such that the highly purified cannabinoid is greater than or equal to 95% (w/w) pure.

A “botanical drug substance” or “BDS” is defined in the Guidance for Industry Botanical Drug Products Draft Guidance, August 2000, US Department of Health and Human Services, Food and Drug Administration Centre for Drug Evaluation and Research as: “A drug derived from one or more plants, algae, or microscopic fungi. It is prepared from botanical raw materials by one or more of the following processes: pulverisation, decoction, expression, aqueous extraction, ethanolic extraction or other similar processes.”

A botanical drug substance does not include a highly purified or chemically modified substance derived from natural sources. Thus, in the case of Cannabis, BDS derived from Cannabis plants do not include highly purified cannabinoids.

The term “microparticle” or “microparticulate” refers to particle between 1 and 1000 μm in size. In the terms of the present invention a microparticulate comprises an active agent such as a cannabinoid in addition to one or more cannabinoids.

### DETAILED DESCRIPTION OF THE INVENTION

### Active Pharmaceutical Ingredients

An object of the invention is to provide improved cannabinoid containing formulations.

There are many known cannabinoids and the formulation according to the present invention comprises at least one cannabinoid selected from the group consisting of: cannabichromene (CBC), cannabichromenic acid (CBCV), cannabidiol (CBD), cannabidiolic acid (CBDA), cannabidivarin (CBDV), Cannabidiol-C1 (CBD-C1) also known as cannabidiorcol, Cannabidiol-C4 (CBD-C4) also known as nor-cannabidiol, cannabidiol-C6 (CBD-C6), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA). This list is not exhaustive and merely details the cannabinoids which are identified in the present application for reference. So far, over 100 different cannabinoids have been identified and these cannabinoids can be split into different groups as follows: Phytocannabinoids; Endocannabinoids; and Synthetic cannabinoids.

The formulation according to the present invention may also comprise at least one cannabinoid selected from those disclosed in Handbook of Cannabis, Roger Pertwee, Chapter 1, pages 3 to 15.

It is preferred that the formulation comprises one or more cannabinoids, which are preferably selected from the group consisting of, cannabidiol (CBD) or cannabidivarin (CBDV), tetrahydrocannabinol (THC), tetrahydrocannabivarin (THCV), cannabigerol (CBG) and cannabidiolic acid (CBDA) or a combination thereof. It is preferred that the formulation comprises cannabidiol (CBD) and/or cannabidivarin (CBDV).

In a further embodiment it is preferred that the formulation comprises at least two cannabinoids. Preferably these cannabinoids are selected from the group consisting of, cannabidiol (CBD), tetrahydrocannabinol (THC), tetrahydrocannabivarin (THCV), cannabigerol (CBG) and cannabidiolic acid (CBDA).

It is preferred that the one or more cannabinoid is present in an amount of from about 0.1 to 30 (% w/v), based on the total composition, preferably from about 5 to 15 (% w/v).

Preferably, the one or more cannabinoid is synthetic or highly purified from its natural source (for example, plant derived recrystallized form). When a highly purified source is used, it is purified such that the one or more cannabinoid is present at greater than 95%, more preferably 98% of the total extract (w/w).

In a further embodiment the one or more cannabinoids are present as a complex mixture or as a botanical drug substance (BDS). When present as such as mixture the major cannabinoid is present in addition to all the other cannabinoid and non-cannabinoid components that are co-extracted with the major cannabinoid. THC BDS and CBD BDS have been characterized in the patent application WO 2007/083098 which is incorporated in its entirety.

In a further embodiment the formulation comprises a mixture of a cannabinoid which is present as a highly purified (>98%) or synthetic form, in combination with a cannabinoid which is present as a complex mixture or a BDS.

The unit dose of cannabinoid in the oral pharmaceutical formulation may be in the range of from 0.001 to 350 mg/mL, preferably 0.1 to 35 mg/mL, more preferably 1 to 20 mg/mL.

### Excipients

In order to produce the microparticulate polymers comprising cannabinoids the following excipients are of importance.

### pH Dependent Release Polymers:

The pH dependent release polymers of the present invention are used to enable release of the active agent at a pH of either pH 6 (intestines) or pH 7 (colon) rather than at an acidic pH (such as occurs in the stomach). Suitable polymers that may be used include: polymethacrylate derivatives (such as a copolymer of methacrylic acid and methacrylate, a copolymer of methacrylic acid and methyl methacrylate or a copolymer of methacrylic acid and ethylacrylate); hypromellose derivatives (such as hydroxypropyl methyl cellulose acetate succinate (HPMCAS) and hydroxypropyl methyl cellulose phthalate (HPMCP)); polyvinylacetate derivatives (such as polyvinyl acetate phthalate (PVAP)); polyvinylether derivatives (such as a copolymer of methyl vinyl ether and maleic anhydride); cellulose derivatives (such as cellulose acetate phthalate (CAP), cellulose acetate terephthalate, cellulose acetate isophthalate, cellulose acetate butyrate (CAB), cellulose acetate trimellitate (CAT), cellulose acetate succinate (CAS), ethyl cellulose, methyl cellulose); shellac, gellan gum, zein, alginic acid, waxes and mixtures thereof.

The polymer HPMCAS and the copolymer of methacrylic acid and methyl methacrylate are preferred. The copolymer of methacrylic acid and methyl methacrylate is known under the tradename Eudragit®. Two forms of Eudragit are known: L100 and S100. The L100 is a copolymer of the two compounds in a 1:1 ratio and the S100 additionally comprises 0.3% sodium laurylsulfate.

### Hydroxypropyl Methylcellulose Acetate Succinate (HPMCAS)

HPMCAS is a cellulose derived polymer containing acetyl and succinoyl groups. It is an enteric polymer which dissolves at a pH range of between 5.5 and 6.5 depending on the ratio of acetyl and succinoyl groups found within the polymer.

It is widely used a solubility enhancer for poorly soluble drugs, solubility enhancement occurs when HPMCAS is formulated into a solid dispersion along with an API.

Three grades of HPMCAS are available; HPMCAS-L, HPMCAS-M and HPMCAS-H, these polymers dissolve at pH 5.5, 6.0 and 6.5 respectively.

HPMCAS was chosen as a suitable carrier due to its regulatory acceptability, available toxicological data, it shares common solvents with cannabinoids, its versatility and most importantly the pH at which the polymer dissolves.

Eudragit L100 (Methacrylic Acid and Methyl Methacrylate Copolymer (1:1))

Eudragit L100 is a copolymer comprised of methacrylic acid and methyl methacrylate in a 1:1 ratio. The ratio of methacrylic acid to methyl methacrylate controls the pH at which the polymer dissolves. Eudragit L100 is designed to release at a pH of 6.0 and above.

It is most commonly dispersed in an aqueous base to be spray coated onto tablets or capsules to give them an enteric coating. It can also be used as a solubility enhancer for poorly water-soluble drugs when formulated into a solid dispersion along with an API.

Eudragit L100 was chosen as a suitable carrier due to its regulatory acceptability, available toxicological data, it shares common solvents with cannabinoids, its versatility and most importantly the pH at which the polymer dissolves.

### Eudragit S100 (Methacrylic Acid and Methyl Methacrylate Copolymer (1:2))

Eudragit L100 is a copolymer comprised of methacrylic acid and methyl methacrylate in a 1:2 ratio. Eudragit S100 is designed to release at a pH of 7.0 and above.

It is most commonly dispersed in an aqueous base to be spray coated onto tablets or capsules to give them a colonic coating. It can also be used as a solubility enhancer for poorly water-soluble drugs when formulated into a solid dispersion along with an API.

Eudragit S100 was chosen as a suitable carrier due to its regulatory acceptability, available toxicological data, it shares common solvents with cannabinoids, its versatility and most importantly the pH at which the polymer dissolves.

### Wetting Agent:

### Poloxamer 188

Poloxamer 188 is an amphiphilic co-polymer that has multifunctionality. It can be used as a solubiliser, emulsifier and also as a wetting agent for solid dispersion formulations. Poloxamer 188 has an HLB value of 29 meaning it is highly hydrophilic.

Poloxamer 188 was chosen as a potential wetting agent because of the positive impact it can upon hydration properties, its previous use in cannabinoid formulations has revealed low levels of incompatibility and because of its regulatory acceptability.

### Other Wetting Agents

Other wetting agents such as those listed below will be interchangeable with Poloxamer P188. These include: poloxamers; polysorbate 80; sodium carbonate; polyethylene glycols (PEG, Mw 1500-20,000); hydrophilic colloids such as acacia, alginates, methycellulose; alcohols; and glycerin.

### Suspending Agents:

### Polysorbate 20 (Tween 20)

Tween 20 is a nonionic surfactant that has multifunctionality. It is formed by the ethoxylation of sorbitol. As the name suggests the ethoxylation process leaves the excipient with 20 repeating units. These repeating units are comprised of polyethylene glycol. Tween 20 is able to act as an emulsifier, wetting agent and also a solubiliser. Tween 20 has an HLB value of 16.7 meaning it is a hydrophilic surfactant.

### Glycerol

Glycerol is a colourless and odourless viscous liquid. It is widely used as a sweetener and humectant in the food and pharmaceutical industry.

### Xanthan Gum

Xanthan gum is commonly used as a food additive and in the pharmaceutical industry as an agent that increases the viscosity of a liquid.

### Antioxidants:

### Alpha Tocopherol

Alpha Tocopherol is a derivative if Vitamin E. It is commonly used as an antioxidant in pharmaceutical formulations.

Alpha Tocopherol was chosen as a potential antioxidant because of its regulatory acceptability, it has already shown to be effective in limiting oxidation in other cannabinoid formulations, it has the advantage that it is already naturally present within the Cannabis plant and that it shares common solvents with cannabinoids.

Butylated Hydroxytoluene (BHT)

BHT is a crystalline antioxidant commonly used in pharmaceutical formulations.

BHT was chosen as a potential antioxidant because of its regulatory acceptability and that it shares common solvents with cannabinoids.

### Butylated Hydroxyanisole (BHA)

BHA is a crystalline antioxidant commonly used in pharmaceutical formulations.

BHA was chosen as a potential antioxidant because of its regulatory acceptability and that it shares common solvents with cannabinoids.

### pH Buffers:

### Sodium Hydroxide

Sodium hydroxide is an alkali commonly used as a pH adjusting agent. It is listed on the FDA inactive ingredients database for use in oral pharmaceutical formulations with a maximum concentration of 8%. The pH of a sodium hydroxide solution is 13 making it a strong alkali. Sodium hydroxide was chosen as an excipient because of its ability to modify the pH of solutions.

### Edetate Calcium Disodium (EDTA)

EDTA is a commonly used as chelating agent in pharmaceutical formulations. A chelating agent “mops” up free radicals therefore enhancing the stability of a pharmaceutical formulation.

EDTA was chosen as a potential chelating agent because of its regulatory acceptability and also that is has previously demonstrated that it improves the stability of cannabinoid-based formulations namely Oral aqueous solutions and Intravenous solutions.

### Phosphate Buffered Saline (PBS)

PBS is a buffer solution comprising of Sodium chloride, Potassium chloride, Disodium phosphate and Monopotassium phosphate. The pH of PBS is 7.4. PBS was chosen because of its ability to modify and buffer the pH of solutions; it is also commonly used in biological research and has its components have good regulatory acceptability.

### Solvents:

### Water

Water was chosen as a cosolvent for the Eudragit based formulations for several different reasons. Literature suggests that the addition of water to the system leads to the formation of more spherical microspheres (Jablan & Jug, 2015.). Spherical shaped microspheres have the advantage that they flow better and that if suspended they do not aggregate as easily. It also gives the option of incorporating water soluble additives into the system. Finally, water is non-toxic.

### Acetone

Acetone was chosen as a solvent for the HPMCAS based formulations. Acetone is only capable of forming a suspension of HPMCAS; however, it does have significant advantages. Acetone has a low boiling point of 56° C. meaning that reducing residual acetone levels to an acceptable value is straightforward. Also, it has an acceptable toxicological profile with it falling outside of the FDA Class 1-3 solvent classification system.

Cellulose polymers are hard to dissolve to yield solutions, more toxic solvents such as DMSO can dissolve HPMCAS however the trouble comes when having to reduce the solvent concentration to acceptable levels.

### Ethanol

Ethanol was chosen as a cosolvent for the Eudragit based formulations. Ethanol is capable of solubilising L100 completely but only forms suspensions of S100. Addition of water to a S100 ethanol suspension yields a clear solution.

Ethanol has a low boiling point of 78° C. meaning that reducing residual ethanol levels to an acceptable value is straightforward. Also, it has an acceptable toxicological profile with it falling outside of the FDA Class 1-3 solvent classification system.

### Example 1: Preferred Formulations

It is preferred that the microparticulate cannabinoid formulation according to the invention is able to minimize cannabinoid metabolism.

Polymeric microspheres have the potential to reduce the metabolism via two different mechanisms, firstly literature suggests that at the correct particle size (between 5-10 μM) polymeric microspheres can be engulfed as a whole particle by the intestinal cell wall therefore protecting the entrapped drug from degradative enzymes.

Secondly controlled release polymers can be used to deliver the entrapped drug to different parts of the GI tract such as the colon; this turn may alter the metabolic profile of the entrapped cannabinoid.

The following represent preferred formulations according to the invention which may be used to prepare cannabinoid microspheres. Here the active agent is provided as cannabidiol, however the microspheres may be produced using any natural or synthetic cannabinoid, their salts or prodrugs.

20% CBD HPMCAS-L 5% P188 Microspheres

| | | |
|---|---|---|
| CBD | 20 | (% w/w) |
| HPMCAS-L | 74.8 | (% w/w) |
| Kolliphor P188 | 5 | (% w/w) |
| Alpha Tocopherol | 0.2 | (% w/w) |

15% HPMCAS-M 5% P188 Microspheres

| | | |
|---|---|---|
| CBD | 15 | (% w/w) |
| HPMCAS-M | 79.8 | (% w/w) |
| Kolliphor P188 | 5 | (% w/w) |
| Alpha Tocopherol | 0.2 | (% w/w) |

20% CBD L100 Microspheres

| | | |
|---|---|---|
| CBD | 20 | (% w/w) |
| Eudragit L100 | 78.28 | (% w/w) |
| Calcium Disodium EDTA | 1.52 | (% w/w) |
| Alpha Tocopherol | 0.2 | (% w/w) |

15% CBD S100 5% P188 Microspheres

| | | |
|---|---|---|
| CBD | 15 | (% w/w) |
| Eudragit L100 | 78.28 | (% w/w) |
| Kolliphor P188 | 5 | (% w/w) |
| Sodium Hydroxide | 1.52 | (% w/w) |
| Alpha Tocopherol | 0.2 | (% w/w) |

15% CBD S100 20% P188 Microspheres

| | | |
|---|---|---|
| CBD | 15 | (% w/w) |
| Eudragit L100 | 63.28 | (% w/w) |
| Kolliphor P188 | 20 | (% w/w) |
| Sodium Hydroxide | 1.52 | (% w/w) |
| Alpha Tocopherol | 0.2 | (% w/w) |

As is described above, the cannabinoid was added at a concentration of 15% and 20% to produce the microspheres, however concentrations may be used of from 0.1% to 30% cannabinoid. The concentration of the cannabinoid will depend on the cannabinoid used and the therapeutic indication for which the formulation is to be used to treat.

Tables 2 to 6 below illustrate example formulations suitable for colonic or enteric release. Here the cannabinoid microspheres described above have been formulated to produce a suspension. The cannabinoids used in these example formulations are cannabidiol (CBD) or a combination of highly purified CBD and a CBD BDS, here there is a mixture of major cannabinoids in the formulation, namely CBD and THC in addition to the other minor cannabinoids and non-cannabinoids which occur in a BDS. Clearly other cannabinoids or combinations of purified and BDS can be utilized to prepare colonic or enteric release formulations.

TABLE 2
Example formulation for 30 mg/mL CBD
Enteric Release (ER) suspension

| | Composition | Compositions |
|---|---|---|
| Component | (% w/w) | (mg/mL) |
| Cannabidiol (CBD) | 3 | 30.00 |
| AQOAT HPMCAS-L | 11.22 | 112.20 |
| Kolliphor P188 | 0.75 | 7.50 |
| Alpha-Tocopherol | 0.03 | 0.30 |
| Glycerol | 20 | 200.00 |
| Xanthan Gum | 0.2 | 2.00 |
| Citric Acid | 0.25 | 2.50 |
| Sodium Phosphate Dibasic | 0.12 | 1.20 |
| Potassium Sorbate | 0.10 | 1.00 |
| Sodium Benzoate | 0.10 | 1.00 |
| Ascorbic Acid | 0.20 | 2.00 |
| Water | Q.S to 100% | Q.S. to 100% |

TABLE 3
Example formulation for 25 mg/mL CBD Colonic
Release (CR) suspension 5% P188

| | Composition | Compositions |
|---|---|---|
| Component | (% w/w) | (mg/mL) |
| Cannabidiol (CBD) | 2.50 | 25.00 |
| Eudragit S100 | 13.00 | 130.00 |
| Kolliphor P188 | 0.75 | 7.50 |
| Alpha-Tocopherol | 0.03 | 0.30 |
| Sodium Hydroxide | 0.25 | 2.50 |
| Glycerol | 20.00 | 200.00 |
| Xanthan Gum | 0.20 | 2.00 |
| Citric Acid | 1 | 10.00 |
| Sodium Phosphate Dibasic | 0.48 | 4.80 |
| Potassium Sorbate | 0.10 | 1.00 |
| Sodium Benzoate | 0.10 | 1.00 |
| Ascorbic Acid | 0.20 | 2.00 |
| Water | Q.S to 100% | Q.S. to 100% |

TABLE 4
Example formulation for 25 mg/mL CBD Colonic
Release (CR) suspension 20% P188

| | Composition | Compositions |
|---|---|---|
| Component | (% w/w) | (mg/mL) |
| Cannabidiol (CBD) | 2.50 | 25.00 |
| Eudragit S100 | 10.75 | 107.50 |
| Kolliphor P188 | 3 | 30 |
| Alpha-Tocopherol | 0.03 | 0.30 |
| Sodium Hydroxide | 0.25 | 2.50 |
| Glycerol | 20.00 | 200.00 |
| Xanthan Gum | 0.20 | 2.00 |
| Citric Acid | 1 | 10.00 |
| Sodium Phosphate Dibasic | 0.48 | 4.80 |
| Potassium Sorbate | 0.10 | 1.00 |
| Sodium Benzoate | 0.10 | 1.00 |
| Ascorbic Acid | 0.20 | 2.00 |
| Water | Q.S to 100% | Q.S. to 100% |

TABLE 5
Example formulation for 24 mg/mL CBD 0.6
mg/mL THC Enteric Release (ER) suspension

| | Composition | Compositions |
|---|---|---|
| Component | (% w/w) | (mg/mL) |
| CBD Pure | 1 | 10.00 |
| CBD BDS | 2 | 20.00 |
| AQOAT HPMCAS-L | 11.22 | 112.2 |
| Kolliphor P188 | 0.75 | 7.50 |
| Alpha-Tocopherol | 0.03 | 0.30 |
| Glycerol | 20 | 200.00 |
| Xanthan Gum | 0.2 | 2.00 |
| Citric Acid | 0.25 | 2.50 |
| Sodium Phosphate Dibasic | 0.12 | 1.20 |
| Potassium Sorbate | 0.10 | 1.00 |
| Sodium Benzoate | 0.10 | 1.00 |
| Ascorbic Acid | 0.20 | 2.00 |
| Water | Q.S to 100% | Q.S to 100% |

TABLE 6
Example formulation for 20 mg/mL CBD 0.5
mg/mL THC Colonic Release (CR) suspension

| | Composition | Compositions |
|---|---|---|
| Component | (% w/w) | (mg/mL) |
| CBD Pure | 0.825 | 8.25 |
| CBD BDS | 1.665 | 16.67 |
| Eudragit S100 | 13.00 | 130.00 |
| Kolliphor P188 | 0.75 | 7.50 |
| Alpha-Tocopherol | 0.03 | 0.30 |
| Sodium Hydroxide | 0.25 | 2.5 |
| Glycerol | 20.00 | 200.00 |
| Xanthan Gum | 0.20 | 2.00 |
| Citric Acid | 1 | 10.00 |
| Sodium Phosphate Dibasic | 0.48 | 4.80 |
| Potassium Sorbate | 0.10 | 1 |
| Sodium Benzoate | 0.10 | 1 |
| Ascorbic Acid | 0.20 | 2 |
| Water | Q.S to 100% | Q.S. to 100% |

### Method of Administration

The preferred formulations as described above in Tables 2 to 5 is suitable for administration as a medicament. Different modes of administration can be utilised with the formulations, these include an oral solution, an oral suspension, a formulation comprising granules, a formulation comprising sprinkles to be mixed with food, a compressed tablet, a mucoadhesive gel, a tablet, a powder, a liquid gel capsule, a solid powder filled capsule, an extrudate, a nasal spray or an injectable formulation.

When provided as a suspension or an oral solution, the formulation will be dispensed in bottles optionally with syringes such that an accurate dose may be provided to the patient based on an amount of cannabinoid (in mg) per weight of patient (in kg).

In addition, the formulation of the invention may be prepared in alternative means such as a spray, a drink or in a small volume such as 30 mL of solution that is administered to the patient before swallowing.

The Examples that follow describe the development of the formulations of the invention which are formulations comprising cannabinoid microspheres. Such formulations are designed to release their active agent in either the intestines (enteric) or in the colon. Enteric or colonic delivery of cannabinoids which are known to undergo rapid metabolism to inactive metabolites in the body provides a novel and surprisingly efficient way of drug delivery.

### Example 2: Selection of Excipients to Produce an Enteric-Release and a Colonic-Release Microparticulate Formulation

### Drug Hydration Studies

In vitro experimentation assessing drug release from a polymer matrix is important to ensure drug release is achieved from a microparticle in vivo.

Polymer films comprising of API, polymer and wetting agents (if applicable) were manufactured using a solvent casting method.

The produced films were then hydrated in a pH 7.0 buffer and drug release from the polymer films was assessed.

Five different polymers were assessed during drug hydration: Eudragit L100; Eudragit S100; HPMCAS-L; HPMCAS-M and HPMCAS-H.

Two different wetting agents, Poloxamer 188 and Tween 20 were also assessed.

Results of experimentation indicated that a wetting agent is required to aid drug release for all polymers except for the Eudragit L100 polymer. Additionally, it was found that Poloxamer 188 is a more effective wetting agent than Tween 20.

Once hydrated the films formed turbid emulsion. The drug release from the HPMCAS-H polymer was poor at differing drug and wetting agent concentrations.

The following drug and wetting agent concentrations were decided upon and taken forward for further development:
20% CBD; HPMCAS-L; 5% P188
15% CBD; HPMCAS-M; 5% P188
20% CBD; Eudragit L100
15% CBD; Eudragit S100; 20% P188

With the inclusion of wetting agent into the polymer matrices for 3 of the 4 polymers there is a risk that drug release may occur at a pH value consistent with stomach pH. The pH of the stomach is approximately 4.0.

Therefore, films at the above drug and wetting agent concentrations were tested for hydration in a buffer with a pH of 4.0. Drug release at this pH was less than 0.5% for all of the polymer systems tested showing that the inclusion of P188 as a wetting agent did not modify the pH at which the polymer matrix should release the drug as is shown in Table 7 below.

TABLE 7
Percentage drug release at intended and gastric pH

| | % Drug release | % Drug release |
|---|---|---|
| Formulation | at intended pH | at gastric pH |
| 20% CBD; HPMCAS-L; 5% P188 | 96 | 0 |
| 15% CBD; HPMCAS-M; 5% P188 | 93 | 0 |
| 20% CBD; Eudragit L100 | 96 | 0.3 |
| 20% CBD; Eudragit S100; 20% P188 | 95 | 0 |

### Antioxidant Screening

It was necessary to include an antioxidant into the CBD/Polymer system as it was observed that the cannabinoid CBE-I was being formed. CBE I is an oxidation derived degradant of CBD which in turn further degrades to CBE II.

3 different antioxidants were screened, all at a concentration of 0.2% w/w:
Alpha-Tocopherol
Butylated Hydroxytoluene
Butylated Hydroxyanisole

These were included in 4 different polymer matrices each with a nominal CBD drug loading of 15%:
HPMCAS-L
HPMCAS-M
Eudragit L100
Eudragit S100

Samples were manufactured and stored at 40° C./75% RH for a period of 28 days.

Results indicated that for both HPMCAS-L and HPMCAS-M an antioxidant is required as the addition of antioxidant also significantly reduced the number of unknown degradants that were formed in the samples.

The samples containing Eudragit L100 and Eudragit S100 behaved differently than the HPMCAS based samples. The addition of the antioxidant reduced the levels of CBE I and CBE II to below the level of quantification over the course of the study, however large quantities of THC were seen in the samples regardless of whether or not an antioxidant was present. The antioxidant had no effect on the formation of THC. This is because the degradation of CBD to THC is an acidic mechanism and not an oxidation mechanism.

From these experiments it was concluded that all four polymer systems would benefit from the addition of an antioxidant.

### Example 3: Method of Manufacture for an Enteric-Release and a Colonic-Release Microparticulate Formulation

Two alternative methods of manufacture for an enteric-release and a colonic-release microparticulate formulation have been developed. Firstly, spray drying which provides a fine powder which can be further formulated into a suspension or tablet and secondly a hot melt extrusion process whereby a granulate is produced which may be used as an additive or sprinkle. The two processes are described in further detail below.

### Spray Drying

It was determined whether it was possible to spray dry formulations comprising HPMCAS-L (Table 2) and Eudragit S100 (Table 4) containing CBD to form dry powders. Both polymers were spray dried with a nominal drug concentration of 15%.

The HPMCAS-L was spray dried with CBD using the following conditions:
Drug concentration: 15%
Solid concentration: 5%
Inlet temperature: 85° C.
Outlet temperature: 55° C.
Aspirator: 75%
Pump: 5%
Solvent: Acetone

The Eudragit S100 was spray dried with CBD using the following conditions:
Drug concentration: 15%
Solid concentration: 3%
Inlet temperature: 100° C.
Outlet temperature: 62° C.
Aspirator: 100%
Pump: 5%
Solvent: Ethanol:Water 50:50 ratio.

The above conditions produced spray dried powders for both polymers tested showing it is possible to create spray dried powders comprising of HPMCAS and CBD and Eudragit S100 and CBD.

Because of the chemical similarities between the different grades on HPMCAS a positive result for HPMCAS-L would indicate a positive result for the other grades. Eudragit S100 and Eudragit L100 also share similar chemical structures which would indicate that spray drying CBD with L100 would give a positive result.

The following configuration spray dryer is preferred:
Two fluid nozzles with 0.7 mm nozzle tip
Drying gas: Nitrogen
Negative pressure mode
Use of High-performance cyclone instead of standard cyclone
Long drying chamber used with waste collection attachment

### HPMCAS Polymers

Spray drying of HPMCAS-L and HPMCAS-M was interchangeable and as such the same process could be used for HPMCAS-L and HPMCAS-M.

Acetone was chosen as the solvent for spray drying due to its ability to solubilise cannabinoids and HPMCAS. Additionally, it is an FDA Class III solvent because of its limited toxicity. In Acetone HPMCAS dissolves to yield a fine suspension.

### Eudragit Polymers

A mixture of Ethanol and 0.5% w/w EDTA solution was chosen as the solvent mix for the spray drying of the Eudragit L100 polymer. Ethanol was chosen as it is a suitable solvent for cannabinoids and Eudragit L100. It is also an FDA Class III solvent because of its limited toxicity. The EDTA was required as it helped to stabilise the final CBD L100 polymer system. The Ethanol and EDTA solution were completely miscible. The solvent mix comprised of an 80:20 ratio of Ethanol to EDTA solution. Further optimisation could be performed to increase the Ethanol content further, a higher Ethanol content is advantageous because it is more volatile than water

A mixture of Ethanol and 0.1M Sodium hydroxide was chosen as the solvent mix for the spray drying of the Eudragit S100 polymer for the reasons stated above. 0.1M NaOH was the stabiliser of choice for the S100 polymer system.

### Application of Spray Dried Formulation

The resulting spray dried powder generated in the experiments above can then be further formulated to provide a pharmaceutically acceptable formulation.

The spray dried powder may be mixed with a solvent such as water or glycerol to produce a suspension which may be administered orally as a solution. The spray dried powder may alternatively be compressed into tablets of filled in capsules to be swallowed by a patient.

### Hot Melt Extrusion

An alternative means of administration of the microparticulate formulation of the invention is provided. Using the technique of holt melt extrusion a microparticulate granule is produced. Such granules may be used as an additive to food as a sprinkle. Such dosage options are of benefit to younger patients and those patients that may have difficulty swallowing a tablet.

Hot melt extrusion is a process which uses heat and pressure to melt the polymer and active agent. It is solvent free and may increase the solubility and bioavailability of an active agent.

The process is as follows:

The polymer and cannabinoid are mixed together. Optionally an antioxidant and/or a distintegrant may be added after this stage. The blend is mixed to form an intermediate powder blend which is then processed through the hot melt extruder. The extrudates are then pelletised and further milled to the required size. A pellet size of 500 μm/250 μm is preferred.

Samples of hot melt extrusion produced sprinkles were tested to determine they would release at their intended pH rather than at gastric pH and all formulations tested released between 93-96% of their active at the intended pH. None released any active at gastric pH.

The stability of the hot melt extruded polymers was tested over a 12 week period and there were no significant increase of CBD related degradants over the time period nor any changes in the particle size.

### Example 4: Stability of an Enteric-Release and a Colonic-Release Microparticulate Formulation

Two different formulations prepared by spray drying and further formulating into a suspension were put into a short-term stability study as described in Table 8 below.

TABLE 8
Formulation and storage conditions for stability testing

| | Formulation with | | |
|---|---|---|---|
| Num- | microparticulates | Time points | Storage |
| ber | containing: | (days) | conditions |
| 1 | 30 mg/mL CBD; HPMCAS-L | 0, 7, 21, 42 | 5° C./25° C./30° C. |
| 2 | 25 mg/mL CBD; Eudragit | 0, 7, 21, 42 | 25° C./40° C. |
| | S100, 20% P188 | | |
| 3 | 25 mg/mL CBD, Eudragit | 0, 7, 21, 42 | 40° C. 75% RH |
| | S100, 5% P188 | | |
| 4 | 24 mg/mL CBD 0.6 mg/mL | 0, 7, 28 | 5° C./30° C. |
| | THC HPMCAS-L | | |

Tests were undertaken at the various time points to determine the following: appearance; cannabinoid assay; differential scanning calorimetry (DSC) and particle size via the dry dispersion method.

In the case of formulation number 4, this formulation contains a mixture of highly purified CBD and CBD BDS. In order to determine the stability of this formulation the concentration of the major cannabinoids in the formulation, namely CBD and THC were determined along with the degradation products.

Tables 9 to 12 below demonstrate the data obtained from the stability study.

TABLE 9
Stability study outcomes of a 30 mg/mL HPMCAS-L Suspension

| | | % of Active | | |
|---|---|---|---|---|
| | Timepoint | 5° C. | 25° C. | 30° C. |
| CBD | Initial | 100.0 | 100.0 | 100.0 |
| | 1 week | 97.5 | 98.4 | 97.4 |
| | 3 week | 100.9 | 99.1 | 98.9 |
| | 6 week | 101.5 | 101.8 | 101.0 |
| CBD-C4 | Initial | 0.3 | 0.3 | 0.3 |
| | 1 week | 0.3 | 0.3 | 0.3 |
| | 3 week | 0.3 | 0.3 | 0.3 |
| | 6 week | 0.3 | 0.3 | 0.3 |
| CBDV | Initial | 0.2 | 0.2 | 0.2 |
| | 1 week | 0.3 | 0.3 | 0.3 |
| | 3 week | 0.3 | 0.3 | 0.3 |
| | 6 week | 0.3 | 0.3 | 0.3 |
| RRT 0.54 | Initial | 0.0 | 0.0 | 0.0 |
| | 1 week | 0.0 | 0.1 | 0.0 |
| | 3 week | 0.0 | 0.0 | 0.0 |
| | 6 week | 0.0 | 0.0 | 0.0 |
| RRT 0.52 | Initial | 0.0 | 0.0 | 0.0 |
| | 1 week | 0.0 | 0.0 | 0.0 |
| | 3 week | 0.0 | 0.0 | 0.0 |
| | 6 week | 0.0 | 0.0 | 0.0 |

TABLE 10
Stability study outcomes of a 25 mg/mL
CBD S100 with 20% p188 Suspension

| | |
|---|---|
| Timepoint | % of Active |

| | (weeks) | 25° C. | 40° C. |
|---|---|---|---|
| CBD | 0 | 100.00 | 100.00 |
| | 1 | 102.40 | 97.74 |
| | 3 | 105.94 | 106.88 |
| | 6 | 105.64 | 105.15 |
| CBD-C4 | 0 | 0.31 | 0.31 |
| | 1 | 0.31 | 0.30 |
| | 3 | 0.33 | 0.33 |
| | 6 | 0.32 | 0.32 |
| CBDV | 0 | 0.31 | 0.31 |
| | 1 | 0.33 | 0.31 |
| | 3 | 0.33 | 0.33 |
| | 6 | 0.33 | 0.33 |
| THC | 0 | 0 | 0 |
| | 1 | 0 | 0 |
| | 3 | 0 | 0 |
| | 6 | 0 | 0 |

TABLE 11
Stability study outcomes of a 25 mg/mL
CBD S100 with 5% p188 Suspension

| | Timepoint | % of Active |
|---|---|---|
| | (weeks) | 40° C. |
| CBD | 0 | 100.00 |
| | 1 | 101.09 |
| | 3 | 99.35 |
| | 6 | 100.13 |
| CBD-C4 | 0 | 0.30 |
| | 1 | 0.29 |
| | 3 | 0.31 |
| | 6 | 0.32 |
| CBDV | 0 | 0.32 |
| | 1 | 0.33 |
| | 3 | 0.32 |
| | 6 | 0.33 |
| THC | 0 | 0.00 |
| | 1 | 0.00 |
| | 3 | 0.00 |
| | 6 | <BLQ |
| CBD-C1 | 0 | 0.05 |
| | 1 | 0.04 |
| | 3 | 0.05 |
| | 6 | 0.04 |

TABLE 12
Stability study outcomes of a 24 mg/mL
CBD 0.6 mg/mL THC HPMCAS-L Suspension

| | |
|---|---|
| Time point | % of Active |

| Assay | (weeks) | 5° C. | 30° C. 65% RH |
|---|---|---|---|
| CBD | Initial | 104.6 | 104.6 |
| | 2 weeks | 105.8 | 105.3 |
| | 4 weeks | 106.6 | 106.7 |
| THC | Initial | 99.2 | 99.2 |
| | 2 weeks | 101.3 | 101.0 |
| | 4 weeks | 102.0 | 102.2 |
| CBE I | Initial | 0.2 | 0.2 |
| | 2 weeks | 0.2 | 0.2 |
| | 4 weeks | 0.2 | 0.2 |
| CBD-C4 | Initial | 0.3 | 0.3 |
| | 2 weeks | 0.3 | 0.3 |
| | 4 weeks | 0.3 | 0.3 |
| CBG | Initial | 1.4 | 1.4 |
| | 2 weeks | 1.4 | 1.4 |
| | 4 weeks | 1.4 | 1.4 |
| CBN | Initial | 0.1 | 0.1 |
| | 2 weeks | 0.2 | 0.2 |
| | 4 weeks | 0.1 | 0.1 |
| CBC | Initial | 2.9 | 2.9 |
| | 2 weeks | 2.9 | 2.9 |
| | 4 weeks | 2.9 | 2.9 |
| OH-CBD | Initial | 0.6 | 0.6 |
| | 2 weeks | 0.6 | 0.6 |
| | 4 weeks | 0.6 | 0.6 |
| CBDV | Initial | 0.8 | 0.8 |
| | 2 weeks | 0.8 | 0.8 |
| | 4 weeks | 0.8 | 0.8 |

The results presented in Tables 9 to 12 demonstrate that over a period of 1 month at the accelerated conditions there are no major increases in the degradants or decreases in the amount of CBD.

In conclusion the formulations comprising microparticles of cannabinoid and a polymer are stable and allow a shelf life of 6 months.

### Example 5: Particle Size of an Enteric-Release and a Colonic-Release Microparticulate Formulation

The different formulations from the short-term stability study as described in Example 4 above were tested to measure the particle size of the microparticles.

In the case the formulation described in Table 15, this formulation contains a mixture of highly purified CBD and CBD BDS. I

Tables 13 to 15 below describe these data.

TABLE 13
Particle size of 30 mg/mL HPMCAS-L Suspension

| | D₁₀ (μm) | | | D₅₀ (μm) | | | D₉₀ (μm) | | |
|---|---|---|---|---|---|---|---|---|---|
| Time point (weeks) | 5° C. | 25° C. | 30° C. | 5° C. | 25° C. | 30° C. | 5° C. | 25° C. | 30° C. |
| 0 | 3.03 | 3.03 | 3.03 | 7.14 | 7.14 | 7.14 | 21.3 | 21.3 | 21.3 |
| 1 | 3.09 | 3.31 | 3.02 | 7.34 | 9.17 | 6.26 | 42.5 | 20.7 | 14.2 |
| 3 | 2.94 | 3.04 | 3.16 | 6.16 | 6.33 | 6.2 | 14.3 | 14.7 | 14.4 |
| 6 | 3.12 | 3.21 | 3.33 | 7.42 | 6.89 | 7.08 | 33.7 | 22.7 | 87.4 |

TABLE 14
Particle size of 25 mg/mL CBD S100 Suspension

| Time | D₁₀ (μm) | | D₅₀ (μm) | | D₉₀ (μm) | |
|---|---|---|---|---|---|---|
| point (weeks) | 25° C. | 40° C. | 25° C. | 40° C. | 25° C. | 40° C. |
| 0 | 3.72 | 3.72 | 9.33 | 9.33 | 20.1 | 20.1 |
| 1 | 3.79 | 4.01 | 9.04 | 10.9 | 21.7 | 48.4 |
| 3 | 3.80 | 3.80 | 8.75 | 9.81 | 18.6 | 36.7 |
| 6 | 3.83 | 3.49 | 8.87 | 9.64 | 18.5 | 23.9 |

TABLE 15
Particle size of 24 mg/mL CBD 0.6
mg/mL THC HPMCAS-L Suspension

| | D₁₀ (μm) | D₅₀ (μm) | D₉₀ (μm) |
|---|---|---|---|
| Time point (weeks) | 25° C. | 25° C. | 25° C. |
| 0 | 4.10 | 11.1 | 29.1 |
| 1 | 4.01 | 10.3 | 24.7 |
| 4 | 3.92 | 10.4 | 26.6 |

As can be seen the particle size of the cannabinoid containing microparticulate formulations did not alter considerably over the course of the stability study meaning that during storage of the formulation there will not be any degradation of the particle size.

### Example 6: Bioavailability of a Colonic-Release Microparticulate Formulation

In order to determine whether the colonic-release (CR) formulations detailed in Example 1 were able to provide suitable bioavailability a PK study using rats was undertaken.

These formulations were compared with a Type I oil-based formulation.

The active used was CBD for the Type I oil-based formulation and the colonic-release and the enteric-release formulations were tested with two different actives; CBD alone or a combination of THC and CBD.

The design of the study was to measure the plasma pharmacokinetics of CBD and THC and their metabolites (hydroxy-CBD, carboxy CBD, hydroxy-THC and carboxy-THC) following oral administration to the rat.

Male han wistar rats (n=3) per group were fasted prior to dosing and fed at 4 hours post dosing.

The sampling times were: 0, 1, 2, 4, 8, 12 and 24 h post-dose. The determination of CBD, THC and their respective metabolites was performed by protein precipitation with reverse phase liquid chromatography with tandem mass spectrometric detection. The LLOQ of CBD was 1 ng/mL and all metabolites had an LLOQ of 0.5 ng/mL.

The human equivalent dose (HED) can be estimated using the following formula:

$$HED = Animal dose ( mg / kg ) multiplied by \frac{Animal {K}_{m}}{Human {K}_{m}}$$

The Km for a rat is 6 and the Km for a human is 37.

Thus, for a human a 10 mg/kg dose in a rat equates to a human dose of about 1.6 mg/kg.

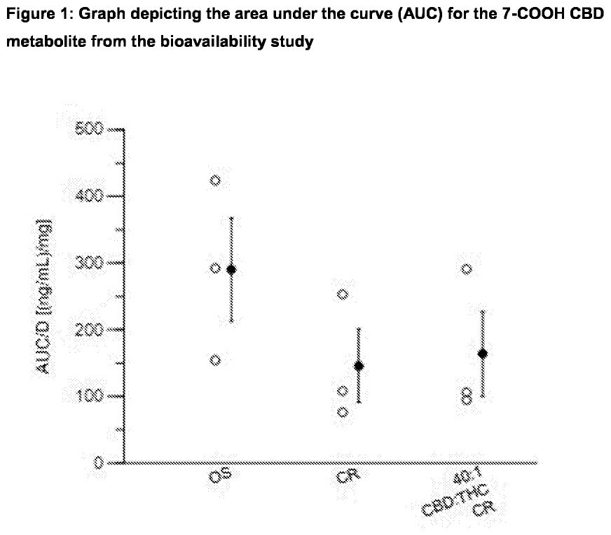

Table 16 details the bioavailability of the different formulations tested and FIG. 1 details the AUC of the non-active metabolite of CBD, 7-COOH CBD. As can be seen in the graph in both the CBD microparticulate suspension and the suspension containing a mixture of highly purified CBD and CBD BDS there is one result which is an outlier suggesting that the actual concentration of 7-COOH CBD was much lower than the mean AUC recorded in the table.

TABLE 16
Estimation of bioavailability (using AUC(0-t) data)

| | Analyte | | | Ratios | | | Analyte | | | Ratios | | |
|---|---|---|---|---|---|---|---|---|---|---|---|---|
| AUC 0-t | | OH- | COOH- | | OH- | COOH- | | OH- | COOH- | | OH- | COOH- |
| (H/ng/ml/mg) | CBD | CBD | CBD | CBD | CBD | CBD | THC | THC | THC | THC | THC | THC |
| Type I (oil-based) | 386 | 61.4 | 290 | 1 | 0.16 | 0.75 | | | | | | |
| CR (CBD) | 338 | 53.8 | 146 | 1 | 0.16 | 0.43 | | | | | | |
| CR (pure CBD + | 187 | 27.6 | 164 | 1 | 0.15 | 0.88 | 1470 | 148 | 218 | 1 | 0.10 | 0.15 |
| CBD BDS) | | | | | | | | | | | | |

The results demonstrate a significant decrease in the amount of the inactive carboxy-CBD metabolite in the colonic-release and the enteric-release formulations in comparison to the Type I oil-based formulation. This is very beneficial as it means that a lower dose of the active can be administered to enable the same effect.

### Example 7: Long-Term Stability of a Preferred Formulation

The suspension containing a mixture of highly purified CBD and CBD BDS in HPMCAS-L was taken forward into a long-term stability study as shown in Table 17. In order to determine the stability of this formulation the concentration of the major cannabinoids in the formulation, namely CBD and THC were determined along with the degradation products.

TABLE 17
Formulation and storage conditions for stability testing

| Formulation with microparticulates | Time points | Storage |
|---|---|---|
| containing: | (weeks) | conditions |
| 25 mg/mL CBD 0.6 mg/mL THC; | 0, 3, 6, 12, 24 | 5° C./25° C./30° C. |
| HPMCAS-L | | |

Tests were undertaken at the various time points to determine the following: appearance; cannabinoid assay; and particle size via the dry dispersion method.

Table 18 below demonstrates the data obtained from the stability study.

TABLE 18
Stability study outcomes of a 25 mg/mL
CBD 0.6 mg/mL THC HPMCAS-L Suspension

| | % of Active | | |
|---|---|---|---|
| Timepoint | 5° C. | 25° C. | 30° C. |

| | | | | |
|---|---|---|---|---|
| CBD | Initial | 100.0 | 100.0 | 100.0 |

| | | | | |
|---|---|---|---|---|
| 3 | week | 100.06 | 101.44 | 101.42 |
| 6 | week | 98.12 | 96.15 | 96.34 |
| 12 | week | 99.96 | 98.56 | 98.96 |
| 24 | week | 99.32 | 98.29 | 97.53 |

| | | | | |
|---|---|---|---|---|
| THC | Initial | 100.00 | 100.00 | 100.00 |

| | | | | |
|---|---|---|---|---|
| 3 | week | 98.19 | 99.74 | 99.83 |
| 6 | week | 98.36 | 97.93 | 97.50 |
| 12 | week | 100.34 | 99.14 | 99.57 |
| 24 | week | 101.12 | 100.17 | 98.62 |

| | | | | |
|---|---|---|---|---|
| CBE I | Initial | 0.30 | 0.31 | 0.30 |

| | | | | |
|---|---|---|---|---|
| 3 | week | 0.31 | 0.31 | 0.31 |
| 6 | week | 0.31 | 0.32 | 0.31 |
| 12 | week | 0.30 | 0.30 | 0.30 |
| 24 | week | 0.31 | 0.32 | 0.30 |

| | | | | |
|---|---|---|---|---|
| OH-CBD | Initial | 0.55 | 0.56 | 0.55 |

| | | | | |
|---|---|---|---|---|
| 3 | week | 0.54 | 0.58 | 0.55 |
| 6 | week | 0.50 | 0.52 | 0.51 |
| 12 | week | 0.56 | 0.58 | 0.56 |
| 24 | week | 0.55 | 0.57 | 0.59 |

| | | | | |
|---|---|---|---|---|
| CBN | Initial | 0.12 | 0.12 | 0.12 |

| | | | | |
|---|---|---|---|---|
| 3 | week | 0.12 | 0.13 | 0.13 |
| 6 | week | 0.10 | 0.10 | 0.10 |
| 12 | week | 0.13 | 0.13 | 0.13 |
| 24 | week | 0.12 | 0.12 | 0.13 |

The results presented in Table 18 demonstrate that over a period of 6 months at differing temperatures there are no major increases in the degradants (CBE-I, OH-CBD, CBN) or decreases in the amount of the major cannabinoids CBD or THC.

In conclusion the formulations comprising microparticles of cannabinoid and a polymer are stable and allow a shelf life of at least 6 months.

### Example 8: Particle Size from Long-Term Study

The formulation from the long-term stability study as described in Example 7 above was tested to measure the particle size of the microparticles.

Table 19 below describes this data.

TABLE 19
Particle size of pure CBD + CBD BDS (25
mg/mL CBD 0.6 mg/mL THC) HPMCAS-L Suspension

| | D₁₀ (μm) | | | D₅₀ (μm) | | | D₉₀ (μm) | | |
|---|---|---|---|---|---|---|---|---|---|
| Time point (weeks) | 5° C. | 25° C. | 30° C. | 5° C. | 25° C. | 30° C. | 5° C. | 25° C. | 30° C. |
| 0 | 3.25 | 3.35 | 3.35 | 7.24 | 7.24 | 7.24 | 17.3 | 17.3 | 17.3 |
| 3 | 3.65 | 3.22 | 3.11 | 8.25 | 6.98 | 6.50 | 18.6 | 16.8 | 15.7 |
| 6 | 3.67 | 3.24 | 3.06 | 8.37 | 6.94 | 6.37 | 18.2 | 17.3 | 15.5 |
| 12 | 3.77 | 3.25 | 3.09 | 8.71 | 6.87 | 6.20 | 19.4 | 18.9 | 14.1 |
| 24 | 3.61 | 3.19 | 3.06 | 7.99 | 6.61 | 6.22 | 17.0 | 15.9 | 15.2 |

As can be seen the particle size of the cannabinoid containing microparticulate formulations did not alter considerably over the course of the stability study meaning that during long-term storage of the formulation there will not be any degradation of the particle size.

1. A microparticulate cannabinoid containing formulation comprising one or more cannabinoids and a pH dependent release polymer.

2. A microparticulate cannabinoid containing formulation according to claim 1, wherein the one or more cannabinoids are taken from the group consisting of: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) and tetrahydrocannabivarinic acid (THCVA).

3. A microparticulate cannabinoid containing formulation according to claim 1, wherein the pH dependent release polymer is taken from the group consisting of: a copolymer of methacrylic acid and methacrylate, a copolymer of methacrylic acid and methyl methacrylate (Eudragit), a copolymer of methacrylic acid and ethylacrylate, hydroxypropyl methyl cellulose acetate succinate (HPMCAS), hydroxypropyl methyl cellulose phthalate (HPMCP), polyvinyl acetate phthalate (PVAP), a copolymer of methyl vinyl ether and maleic anhydride, cellulose acetate phthalate (CAP), cellulose acetate butyrate (CAB), cellulose acetate trimellitate (CAT), cellulose acetate succinate (CAS), ethyl cellulose, methyl cellulose, shellac, gellan gum, zein, alginic acid and waxes.

4. A microparticulate cannabinoid containing formulation according to claim 3, wherein the pH dependent release polymer is HPMCAS or Eudragit.

5. A microparticulate cannabinoid containing formulation according to claim 4, wherein the pH dependent release polymer is taken from the group consisting of: HPMCAS-L; HPMCAS-M; HPMCAS-H; Eudragit S100; Eudragit L100.

6. A microparticulate cannabinoid containing formulation according to claim 1, further comprising one or more wetting agents.

7. A microparticulate cannabinoid containing formulation according to claim 6, wherein the one or more wetting agents are taken from the group consisting of: poloxamers; poloxamer 188; and sodium carbonate.

8. A microparticulate cannabinoid containing formulation according to claim 1, further comprising one or more suspending agents.

9. A microparticulate cannabinoid containing formulation according to claim 8, wherein the one or more suspending agents are taken from the group consisting of: polysorbate 20; glycerol; and xanthan gum.

10. A microparticulate cannabinoid containing formulation according to claim 1, further comprising one or more pH buffers.

11. A microparticulate cannabinoid containing formulation according to claim 10, wherein the one or more pH buffers are taken from the group consisting of: citric acid; sodium phosphate dibasic; sodium hydroxide; and phosphate buffered saline.

12. A microparticulate cannabinoid containing formulation according to claim 1, further comprising one or more preservatives.

13. A microparticulate cannabinoid containing formulation according to claim 12, wherein the one or more preservatives are taken from the group consisting of: potassium sorbate; and sodium benzoate.

14. A microparticulate cannabinoid containing formulation according to claim 1, further comprising one or more antioxidants.

15. A microparticulate cannabinoid containing formulation according to claim 14, wherein the one or more antioxidants are taken from the group consisting of: butylated hydroxyltoluene; butylated hydroxylanisole; alpha-tocopherol (Vitamin E); ascorbyl palmitate; ascorbic acid; sodium ascorbate; ethylenediamino tetraacetic acid; cysteine hydrochloride; citric acid; sodium citrate; sodium bisulfate; sodium metabisulfite; lecithin; propyl gallate; sodium sulfate; monothioglycerol and mixtures thereof.

16. A microparticulate cannabinoid containing formulation according to claim 1, further comprising one or more solvents.

17. A microparticulate cannabinoid containing formulation according to claim 16, wherein the one or more solvents is taken from the group consisting of: water; ethanol and acetone.

18. A microparticulate cannabinoid containing formulation according to claim 1, wherein the one or more cannabinoids are present in an amount of from about 10 to 50 wt %, based on the pharmaceutical formulation, preferably from about 10 to 30 wt %, more preferably from about 20 to 30 wt %.

19. A microparticulate cannabinoid containing formulation according to claim 1, wherein the formulation is an oral dosage form selected from the group consisting of: a mucoadhesive gel; a tablet; a powder; a liquid gel capsule; a solid capsule; an oral solution; an oral suspension; a granulate; and an extrudate.

20. A microparticulate cannabinoid containing formulation according to claim 1, for use in the treatment of conditions requiring the administration of a neuroprotectant or anti-convulsive medication.

21. A microparticulate cannabinoid containing formulation for use according to claim 20, for use in the treatment of seizures.

22. A microparticulate cannabinoid containing formulation for use according to claim 20, for use in the treatment of Dravet syndrome, Lennox Gastaut syndrome, myoclonic seizures, juvenile myoclonic epilepsy, refractory epilepsy, schizophrenia, juvenile spasms, West syndrome, infantile spasms, refractory infantile spasms, tuberous sclerosis complex, brain tumours, neuropathic pain, Cannabis use disorder, post-traumatic stress disorder, anxiety, early psychosis, Alzheimer's disease, and autism.

23. A method of preparing a microparticulate cannabinoid containing formulation according to claim 1, comprising spray drying the formulation.

24. A method of preparing a microparticulate cannabinoid containing formulation according to claim 1, comprising:
i) Preparing a mixture of the cannabinoid and pH dependent release polymer;
ii) Producing an intermediate powder blend;
iii) Processing the intermediate powder blend through a hot melt extruder
iv) Pelleting the extrudates; and
v) Milling the pellets to 250-500 μm.

25. A method according to claim 24, wherein an antioxidant is added after step (i).

26. A method according to claim 24, wherein a disintegrant is added after step (i).

27. A method of treating a subject comprising administering a microparticulate cannabinoid containing formulation according to claim 1 to the subject.

28. A method according to claim 27, wherein the subject is a human.
